(19) United States
(12) Patent Application Publication
(10) Pub. No.: US 2022/0387794 A1
(43) Pub. Date: Dec. 8, 2022

(54) Device and Method to Acutely Control Colonic Function, Fecal Propulsion, and Defecation

(71) Applicant: Motility Medical Inc., Saint Petersburg, FL (US)

(72) Inventors: Amol N. Soin, Dayton, OH (US); Jennifer S. French, Saint Petersburg, FL (US); Eric A. Schepis, Alpharetta, GA (US); Natalia Alexeeva, Alpharetta, GA (US)

(21) Appl. No.: 17/833,445

(22) Filed: Jun. 6, 2022

Related U.S. Application Data
(60) Provisional application No. 63/197,577, filed on Jun. 7, 2021.

(51) Int. Cl. A61N 1/36; A61N 1/04; A61N 1/05; A61N 1/372

(52) U.S. Cl. CPC A61N 1/36007; A61N 1/0484; A61N 1/0452; A61N 1/0456; A61N 1/36031; A61N 1/0509; A61N 1/37235

(57) ABSTRACT

A system for fecal propulsion includes one or more sensors, a stimulator, one or more pairs of electrodes, a controller and a programming device. The sensors detect at least one marker of colonic activity from the enteric, autonomic and/or somatic nervous system. The stimulator generates energy of a particular waveform, intensity and/or time-course to modulate targeted neural circuitry with stimulation parameters being adaptable and dynamic to accommodate the various types of modulated tissues. The electrodes deliver stimulation to the enteric, autonomic and/or somatic nervous systems to control fecal propulsion. The controller and programing device change the stimulation parameters in real-time based on sensor feedback to acutely drive the circuitry from rest to productive defecation. The stimulation waveforms can be simultaneously or sequentially delivered to condition some tissues, while modulating others. The system is adaptive and can be driven through a data analytic repository and machine learning algorithm.

### CROSS REFERENCE TO RELATED APPLICATIONS

I hereby claim benefit under Title 35, United States Code, Section 119(e) of U.S. provisional patent application Ser. No. 63/197,577 filed Jun. 7, 2021. The 63/197,577 application is hereby incorporated by reference into this application.

### STATEMENT REGARDING FEDERALLY SPONSORED RESEARCH OR DEVELOPMENT

Not applicable to this application.

### BACKGROUND

The described example embodiments generally relate to acutely controlling colonic function for fecal propulsion and defecation.

The bowel is controlled by a complex interaction of nervous system activity that collectively modulates sensation and reflexes, colonic and rectal peristalsis, internal- and external anal sphincter tone and the puborectalis muscle (for explanation of the anatomy and physiology, please refer to the appendix). These functions are hosted by the enteric-, autonomic- and somatic nervous systems, and are gated by their activity levels and time-course. Disruption of this ensemble (i.e. injury, disease, aging or other) may lead to chronic constipation and/or neurogenic bowel.

Chronic constipation is a gastrointestinal disorder marked by infrequent bowel movements and can last for weeks or longer. In general, it occurs when stool moves too slowly through the digestive tract. Neurogenic bowel is the loss of normal bowel function due to nerve injury or disease, and is prevalent in spinal cord injury, multiple sclerosis, amyotrophic lateral sclerosis, and stroke. The symptoms of neurogenic bowel are varied and may include the inability to sense when the bowel is full, loss of colonic motility and peristalsis, or loss of sphincter control.

Spinal cord injury demonstrates how the disruption of the bowel's control circuitry can lead to chronic constipation. It also underscores the complexity of the control circuitry and need for adaptable solutions. That is, injuries sustained to different levels of the spinal cord causes chronic constipation that are hosted by different mechanisms. Injuries sustained to neurological segments above the spinal cord (i.e., reflexic bowel) disrupts the descending inhibitory control circuitry used to voluntarily relax the external anal sphincter and puborectalis muscles, promoting stool retention and chronic constipation. Despite this, the neural connections within the colonic wall (enteric nervous system) and between the spinal cord and colon (parasympathetic system) are still intact, enabling reflexive coordination of colonic motility and fecal propulsion. Incidentally, intraluminal electromyographic recordings of the colonic wall in persons with reflexic bowel demonstrate higher basal levels of activity compared to able-bodied controls, but the activity is arhythmic and does not change with gastric loading.

Injuries sustained to the sacral neurological segments of the spinal cord or cauda equina (i.e. areflexic bowel) however, cause a flaccid external anal sphincter (loss of pudendal nerve control) and disrupts the sacral parasympathetic outflow to the descending colon. The sacral parasympathetic activity is ordinarily responsible for the propulsive waves that propel stool over long distances, and its disruption promotes constipation. Lastly, colonic electromyogram signals are largely absent in persons with areflexic bowel.

The treatments for chronic constipation and neurogenic bowel are largely ineffective and are challenged by the complexity and variability of the bowel control system. There are currently no commercially available devices that are approved to treat chronic constipation. Despite this, human feasibility studies have been used to investigate the effects of transabdominal electrical and magnetic stimulation paradigms on bowel control in persons with spinal cord injury. These studies similarly use feedforward stimulation paradigms that cannot adapt to the dynamic state (activity levels and time-course) of the bowel control ensemble or to selectively modulate (excite, inhibit or block) its part(s). Likewise, they only demonstrate marginal changes in defecation frequency and total bowel care time after weeks-to-months of stimulation, or may be effective in persons with tetraplegia, but not paraplegia, persons with other types of neurogenic bowel, or able-bodied persons with chronic constipation.

### Colon Anatomy:

The colon can be imagined as a compliant tube that is about 160 cm in length and takes a clockwise circumferential course through the abdomen. It is part of the large intestine and is bound by the ileocecal sphincter at its origin and by the internal anal sphincter (smooth muscle) at the perineum. The colon consists of multiple parts (ascending colon; transverse colon; descending colon; sigmoid colon) and serves to extract water and salt from its contents. Importantly, the colon also advances its contents (or stool) towards the rectum and external anal sphincter (striated muscle) in preparation for defecation.

The internal anal sphincter, external anal sphincter and puborectalis muscle work in concert to maintain fecal continence and to enable defecation. The puborectalis muscle loops around the proximal rectum and maintains a near 90▪ anorectal angle by tethering the rectum toward the pubis. All three structures are contracted to maintain fecal continence and are relaxed to enable defecation.

The colon is innervated by a complicated network of internal and external nervous structures. The extrinsic innervation of the colon modulates reflexive and voluntary control of the colon and external anal sphincter, and peristalsis. The colon is innervated by parasympathetic (vagus; pelvic), sympathetic (superior and inferior mesenteric; hypogastric) and somatic nerves (pudendal).

### Bowel Anatomy and Physiology:

In able-bodied persons, the bowel is controlled by an ensemble of voluntary and involuntary nervous activity that involves multiple sub-systems (i.e. enteric-, autonomic- and somatic nervous systems, including cognition). Moreover, the activity levels and timing between and within each sub-system are coordinated, entrained, and gated.

Defecation occurs after stool has been advanced into the rectum by colonic motility, and then ejected from the rectum and past the external anal sphincter. Colonic motility, the internal anal sphincter and fecal storage are largely controlled by involuntary mechanisms (enteric, parasympathetic, sympathetic). Ejection of stool from the rectum requires both voluntary and involuntary control (somatic, cognition), including the tightening of the abdominal musculature to increase intraabdominal pressure, and the relaxation of the external anal sphincter and puborectalis muscle.

Colonic motility occurs as 3 different phases: 1. Individual segmental contractions; 2. Organized groups of contractions (migrating or nonmigrating), and 3. Special propulsive (giant migrating contractions) waves of peristalsis that propel stool over long distances and into the rectum. In healthy able-bodied persons, colonic transport is expected to take about 12-to-30 hours from the ileocecal valve to the rectum.

The enteric nervous system is largely responsible for local segmental control of the colon. It is contained within the colon itself, and includes Auerbach's—(intramuscular myenteric) and Meissner's plexuses (submucosa). The enteric nervous system coordinates much of the colonic wall movement, which mixes and advances stool short-distances through the colon. When the intestinal wall is stretched or dilated, the nerves of the myenteric plexus cause constriction of the colonic muscles proximal to the dilation site, and those distal to the dilation site to relax, propelling its contents distally towards the rectum.

The parasympathetic system is responsible for the propulsive waves that propel stool over long distances within the colon. The vagus nerve provides parasympathetic innervation to the gut from the esophagus to the splenic flexure of the colon, including the ascending and transverse colon segments. The pelvic nerve carries pelvic parasympathetic fibers from sacral spinal cord levels S2-S4 to the descending colon and rectum. Parasympathetic control is hosted by reflexes that are initiated by chemical stimulation or mechanical dilation of the colon, rectum and anal canal. That is, afferent activity is relayed from the sensory afferents of the colon, rectum or anal canal, to the sacral spinal cord segments, and back to the colon along the vagus or pelvic nerve (S2-S4).

Ejection of stool and defecation occurs when the internal anal sphincter, external anal sphincter and puborectalis muscle are relaxed. The internal anal sphincter is under involuntary control and is innervated by both parasympathetic and sympathetic (superior and inferior mesenteric; hypogastric) fibers. Activation of the parasympathetic fibers causes the sphincter to relax, while activation of the sympathetic fibers cause the sphincter to tighten. The external anal sphincter and puborectalis muscle are under voluntary control (pudendal nerve) and are inhibited during defecation.

Fecal continence is achieved by the simultaneous activation of the sympathetic and pudendal nerves. Activation of the sympathetic and pudendal nerves causes constriction of the internal- and external anal sphincters, and tightening of the puborectalis muscles.

### Colonic Function and Defecation in Neurogenic Bowel:

Neurogenic bowel is described as a loss of normal bowel function due to nerve injury or disease. It is prevalent in spinal cord injury, multiple sclerosis, amyotrophic lateral sclerosis, spina bifida, and stroke. The symptoms of neurogenic bowel are varied and may include the inability to sense when the bowel is full, loss of colonic motility and peristalsis, or loss of sphincter control.

There are generally 2 types of neurogenic bowel in spinal cord injury: Reflexic and Areflexic. Reflexic, or Upper Motor Neuron (UMN) bowel, occurs in persons with injuries above the sacral segments of the spinal cord. In these persons, the spinal cord injury disrupts the descending inhibitory control mechanisms used to relax the external anal sphincter and control the puborectalis muscles, thus promoting stool retention. Despite the loss of voluntary control, the connections between the spinal cord and colon are still intact, allowing the reflexive coordination of colonic motility. Areflexic, or Lower Motor Neuron (LMN) bowel, is caused by injuries to the sacral segments of the spinal cord or cauda equina. Persons with areflexic bowel have a flaccid external anal sphincter and do not have spinal cord mediated colonic motility.

### SUMMARY

Some of the various embodiments of the present disclosure relate to a system that can facilitate fecal propulsion and defecation in persons with chronic constipation and neurogenic bowel by sensing physiological variates (e.g., nervous, mechanical, acoustic, etc.) and delivering adaptable electrical stimulation to acutely control colonic motility, fecal propulsion and defecation. The system includes one or more sensors, a stimulator, one or more pairs of electrodes, a controller and a programming device. The one or more sensors detect at least one marker of colonic activity from the enteric, autonomic and/or somatic nervous system. The stimulator generates electrical or electromagnetic energy of a particular waveform, intensity and/or time-course to modulate targeted neural circuitry with stimulation parameters being adaptable and dynamic to accommodate the various types of modulated tissues (e.g., sensory afferents, autonomic efferents, colonic musculature). The one or more pair of electrodes deliver stimulation to the enteric, autonomic and/or somatic nervous systems to control colonic motility, fecal propulsion, and defecation. The controller and programing device change the stimulation parameters (e.g., waveform, frequency, amplitude, duration, etc.) in real-time based on sensor feedback to acutely drive the circuitry from rest to productive defecation. The stimulation waveforms can be simultaneously or sequentially delivered to condition (e.g., facilitate, inhibit, mask, block) some tissues, while modulating others. The system is adaptive and can be driven through a data analytic repository and machine learning algorithm.

In certain aspects the present disclosure is directed to a system used to facilitate defecation in persons with reflexic bowel by pacing the disorganized, arhythmical sacral parasympathetic activity that is responsible for colonic propulsion. A sensor records colonic electromyogram signals and uses them as feedback to inform stimulation parameters. The stimulation drives reflexive, coordinated parasympathetic activity along the entire length of the bowel by activating the low-threshold sensory afferents that innervate the rectum and colon. The activation of sensory afferents in the rectum and bowel reflexively activates sacral parasympathetic outflow and colonic contraction. Other sensors (e.g., goniometer, strain gauges, accelerometers, gyroscopes) of the system are used to mark bowel activity and help drive sacral parasympathetic activity, including those that measure intestinal movements or sounds caused by the movement, electrocardiogram signals, respiratory patterns, impedance and galvanic skin response.

In certain aspects the present disclosure is directed to a system used to facilitate defecation in persons with neurogenic bowel having unreliable spinal reflexes by directly stimulating the sacral parasympathetic efferent fibers, enteric fibers and intestinal wall musculature, without relying on spinal reflexes. The system delivers a stimulation paradigm that includes multi-pulse trains of electrical stimulation, delivered in a burst waveform, to the hepatic or splenic fixtures and/or to the sigmoidal and rectosigmoid junctions. The timing of the bursts and the parameters (e.g., amplitude, frequency, duration, etc.) of the multi-pulse stimuli within each burst are informed by sensed activity such as colonic electromyography, acoustic signs of mechanical contractions, electrocardiogram signals, impedance, galvanic skin response and/or respiration.

In certain aspects, when facilitating defecation in persons with neurogenic bowel having unreliable spinal reflexes, an alternative stimulation approach is used by the system. The alternative stimulation approach includes overlaying the burst stimulation waveform used to modulate nervous and muscular tissue with a second stimulation waveform that conditions the parasympathetic and enteric effector fibers and colonic musculature. The second conditioning stimulation waveform helps to control the atrophied (reduced activation threshold) or super-sensitive (increase activation threshold) excitable tissues during modulation. The second conditioning stimulation waveform is delivered through a same electrode pair as delivers the first burst stimulation waveform or through an independently controlled second electrode pair.

There has thus been outlined, rather broadly, some of the embodiments of the present disclosure in order that the detailed description thereof may be better understood, and in order that the present contribution to the art may be better appreciated. There are additional embodiments that will be described hereinafter and that will form the subject matter of the claims appended hereto. In this respect, before explaining at least one embodiment in detail, it is to be understood that the various embodiments are not limited in its application to the details of construction or to the arrangements of the components set forth in the following description or illustrated in the drawings. Also, it is to be understood that the phraseology and terminology employed herein are for the purpose of the description and should not be regarded as limiting.

To better understand the nature and advantages of the present disclosure, reference should be made to the following description and the accompanying figures. It is to be understood, however, that each of the figures is provided for the purpose of illustration only and is not intended as a definition of the limits of the scope of the present disclosure. Also, as a general rule, and unless it is evidence to the contrary from the description, where elements in different figures use identical reference numbers, the elements are generally either identical or at least similar in function or purpose.

### BRIEF DESCRIPTION OF THE DRAWINGS

FIG. 1 is a diagram of a system to acutely control colonic function, fecal propulsion, and defecation in accordance with an example embodiment.

FIG. 2 is an example configuration of a controller usable with the system to acutely control colonic function, fecal propulsion, defecation.

FIG. 3 is an example configuration of the system to acutely control colonic function, fecal propulsion, and defecation in relation to a human user of the system.

FIG. 4 is an example of a handheld embodiment of the system to acutely control colonic function, fecal propulsion, and defecation.

FIG. 5 is another example embodiment of the system to acutely control colonic function, fecal propulsion, and defecation.

FIG. 6 illustrates a raw data trace demonstrating an exemplary sensing and stimulation paradigm to acutely modulate the bowel in persons with constipation.

### DETAILED DESCRIPTION

### A. Overview

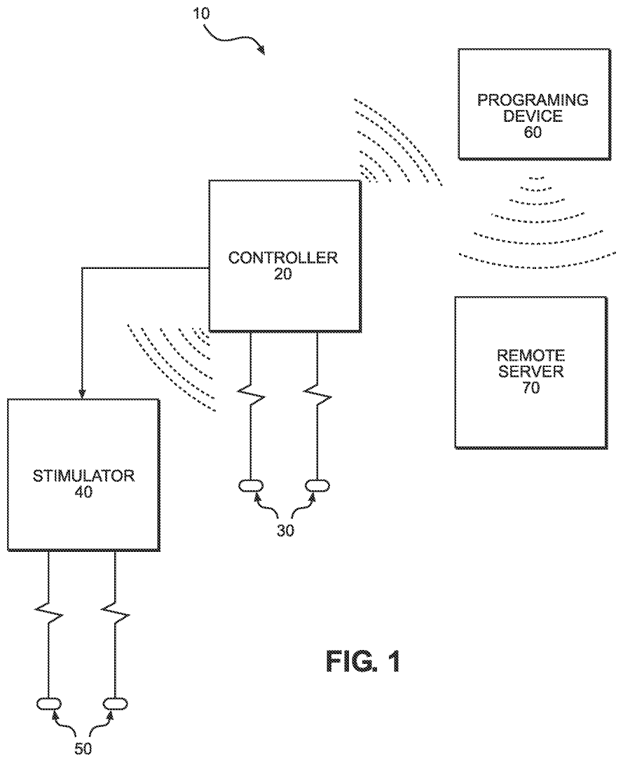

With reference to FIGS. 1-5, a system 10 to acutely control colonic function, fecal propulsion, and defecation in accordance with an example embodiment. The system 10 generally includes a controller 20, one or more sensors 30, and a stimulator 40, one or more pairs of electrodes 50, and a programming device 60.

In certain aspects, the system 10 for fecal propulsion includes a controller 20 executing instructions stored in a memory 23 as well as one or more sensors 30 that are communicatively coupled to the controller 20. The one or more sensors 30 sense at least one marker of colonic activity from one or more of: the enteric nervous system, the autonomic nervous system, and the somatic nervous system of a patient. The system 10 additionally includes a stimulator 40 that is communicatively coupled to the controller 20 and one or more pairs of electrodes 50 that are operatively coupled to the stimulator 40. The controller 20 directs the stimulator 40 to deliver stimulation, via the one or more pairs of electrodes 50, to one or more of the: the enteric nervous system, the autonomic nervous system, and the somatic nervous system of a patient, responsive to the at least one marker of colonic activity to produce fecal propulsion within the patient.

In certain aspects, the one or more sensors 30 additionally sense a physiological variate different from the at least one marker of colonic activity and the controller 20 directs the stimulator 40 to deliver stimulation responsive to both the physiological variate and the at least one marker of colonic activity. The physiological variable can include one or more of: intestinal movements, sounds caused by intestinal movements, electrocardiogram signals, respiratory patterns, muscle tone, autonomic tone, pressor movements, relaxation movements, gross movements, impedance and galvanic skin response.

In certain aspects, the stimulation is in the form of electric or electromagnetic energy. Further, in certain aspects, the stimulation is delivered as a stimulation waveform. The stimulation waveform is determined by controller 20 based on a predetermine algorithm that is responsive to the at least one marker of colonic activity. The predetermined algorithm can comprise a machine-learning algorithm based on historical operating data of the system. In certain aspects, the stimulation comprises at least two different waveforms delivered by the one or more pairs of electrodes; the at least two different waveforms can be delivered simultaneously or sequentially. A first of the at least two different waveforms can condition one or more of the enteric nervous system, the autonomic nervous system, and the somatic nervous system, and a second of the at least two different waveforms modulates nervous and/or muscular tissue.

In certain aspects, at least one pair of the one or more pairs of electrodes 50 comprise external electrodes that are placed on the skin of a patient. The external electrodes can be maintained in position through use of a wearable garment. In certain aspects, at least one pair of the one or more pairs of electrodes are implanted within the patient.

In certain aspects, the system for fecal propulsion additionally includes a programming device 60 that is in direct or remote, wired or wireless communication with the controller 20. The programming device 60 enables adjustment of one or more stimulation parameters that define the stimulation. The programming device 60 receives feedback from one or more of: the one or more sensors, the stimulator and the one or more pairs of electrodes.

### B. Controller

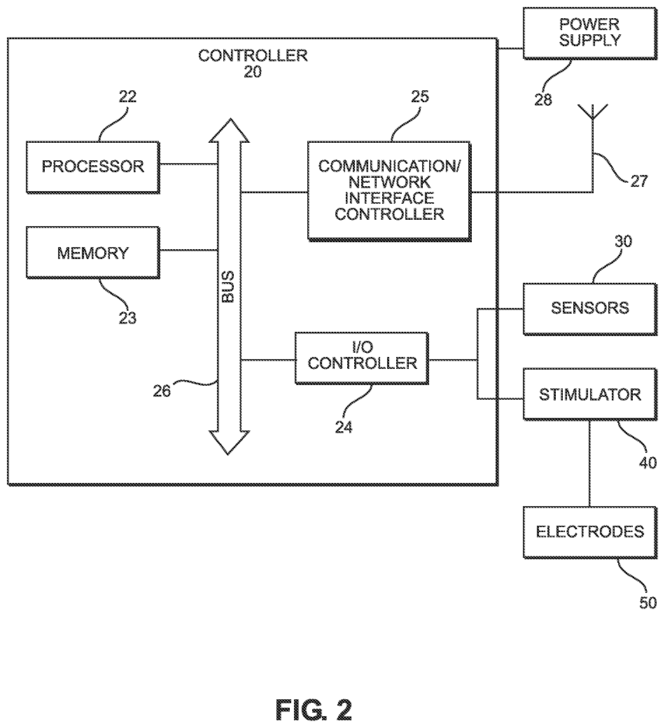

Referring to FIG. 2, an example of a controller 20 includes the components of a processor 22, a memory 23 (e.g., RAM, ROM, EEPROM, NVRAM, flash memory, etc.), an I/O controller 24, a communication/network interface controller (NIC) 26, and a bus 26. However, fewer, additional and/or different components suitable to achieving the desired functionality can also be used.

The processor 22 can be any circuit configured to process information and can include any suitable analog or digital circuit. The processor 22 can also include a programmable circuit that executes instructions. Examples of programmable circuits include microprocessor, microcontrollers, application specific integrated circuits (ASIC), programmable gate arrays (PLA), field programmable gate arrays (FPGA) or any other processor or hardware suitable for executing instructions. In various embodiments, the processor 22 can be a single unit or a combination of two or more units. If the processor includes two or more units, the units can be physically located in a single controller in separate devices.

The memory 23 can include volatile memory such as random access memory (RAM) and non-volatile memory such as read only memory (ROM), electrically erasable programmable read only memory (EEPROM), flash memory, non-volatile random access memory (NVRAM0, magnetic memory, optical memory, or any other suitable memory technology.

The I/O controller 24 is circuitry that monitors operation of the controller 20 and peripheral or external devices such as the one or more sensors 30 and the stimulator 40. The I/O controller 24 also manages the flow of data between the controller 20 and the peripheral devices and frees the processor 22 from details associated with monitoring and controlling the peripheral devices. Examples of other peripheral or external devices with which the I/O controller 24 can include external storage devices, monitors, keyboards, pointing devices, external computing devices, and other remote devices.

The NIC 25 is in electrical communication with an antenna 27 and provides wireless communication between the controller 20 and remote devices (e.g., remote server 70). Communication code stored in memory 23 controls communication through the antenna 27 including processing data embodied in signals received through the antenna 27 and preparing data to be transmitted to remote devices through the antenna 27. Communication can occur according to any wireless transmission technique including Bluetooth and cellular standards (e.g., DMA, GPRS, GSM, 2.5G, 3G, 3.5G, 4G), Wi Gig, IEEE 802.11a/b/g/n/ac, IEEE 802.16 (e.g., WiMax). The NIC 25 can also provide wired communication between the controller 20 and remote devices through wired connections using any suitable port and connector for transmitting data and according to any suitable wired communication standards such as RS 232, USB, FireWire, Ethernet, MIDI, eSATA, or thunderbolt.

The bus 26 includes conductors or transmission lines providing a path to transfer data between the components of the controller 20. The bus 26 typically comprises a control bus, address bus and data bus. However, any bus or combination of buses suitable to transfer data

In certain embodiments, the controller 20 is independently housed while in other embodiments the controller 20 is housed with one or more of the stimulator 40 and the programming device 60. Power to the controller 20, the one or more sensors 30, the stimulator 40, the electrodes 50 and/or the programming device 60 can be provided by one or more power supplies 28 internal power sources (e.g., battery, rechargeable battery) and/or an external power source (e.g., household electric current).

Within the context of the system 10 to acutely control colonic function, fecal propulsion, and defecation, the controller 20 coordinates physiological signals obtained from from the one or more sensors 30 with the stimulation paradigm (e.g., waveforms, parameters and channels), which are programmed into the controller 20 and/or stimulator 40 via the programming device 60. The controller 20 can additionally operate to recognize adverse events based on the physiological signals. For example, the controller 20 can recognize patterns of autonomic dysreflexia (AD) and respond thereto by terminating stimulation and alerting the user to the detected condition. AD is an abnormal, overreaction of the involuntary (autonomic) nervous system to stimulation; the reaction can include change in heart rate, excessive sweating, high blood pressure, muscle spasms, and skin color changes.

### C. Sensor

The one or more sensors 30 are in wired or wireless communication with the controller 20 and are used to measure physiological variates. In certain embodiments, one or more of the sensors 30 are implanted within the body while in other embodiments all sensors 30 are external to the body. Further, in certain embodiments, one or more of the sensors 30 are incorporated into the construct of one or more of the electrodes 50 while in other embodiments the one or more sensors 30 are provided in an individual, independent sensor construct or a multi-sensor construct where one or more of the same or different types of sensors are combined in a unit. In certain embodiments, the one or more sensors 30 provide continuous feedback representative of their measured physiological variate while in other embodiments, the one or more sensors 30 are scheduled by the controller 20 to provide feedback representative of their measured physiological variate at pre-determined times.

The one or more sensors 30 can sense one or more of electrical biopotentials, kinematic, kinetic, and/or acoustic (e.g., vibratory) signals but are not limited thereto. The physiological variates measured by the one or more sensors 30 can include, but are not limited to, intestinal movements (e.g., electromyography signals originating from the bowel musculature and/or neural control circuitry), sounds caused by intestinal movements, electrocardiogram signals, respiratory patterns, muscle tone and/or autonomic tone, colonic movement, pressor and/or relaxation movements, gross movements, impedance and galvanic skin response.

The types of one or more sensors 30 used to measure physiological variates can include, but are not limited to, a goniometer (used to measure the range of motion in a joint), a strain gauge (used to measure the ratio of resistance change of a material body dependent upon the force put onto it), an accelerometer (used to measure vibration or acceleration), a gyroscope (used to measure rotational motion), an audio pickup transducer (used to measure/sense vibrations), a biopotential ampflier (used for measuring/detecting bio-electrical signals), and electrodes (used to measure electric activity in the body).

Sensor feedback respresentative of a measured physiological variate is used to inform an algorithm executed by the controller 20 to orchestrate stimulation via the stimulator 40 and the one or more pairs of electrodes 50. The sensor feedback respresentative of a measured physiological variate can also be used by the controller 20 to assure patient safety such as recognizing, for example, the occurrence of AD.

An example placement of the one or more sensors 30 includes placement of over the entire abdominal cavity (e.g., from below the ribs down to the pelvis) to enable the recording of surface electromyogram signals for measurement of bowel activity (e.g., intestinal movements or sounds caused by those movements); a sensor 30, in the form of a topical ultrasound, can additionally or alternatively be utilized to visually watch contractions of the intestines. Another example placement of the one or more sensors 30 includes placement of one or more electrode sensors on the skin surface of the anterior abdomen proximate one or more of the transverse colon, descending colon, cecum, and rectum to sense one or more markers of colonic activity from the enteric, autonomic and somatic nervous systems; a sensor in the form of a topical ultrasound can additionally or alternatively be used to visually observe the one or more markers.

### D. Stimulator

The stimulator 40 is in communication with both the controller 20 and the one or more pairs of electrodes 50. The stimulator 40 functions to generate, and deliver via the one or more pairs of electrodes 50, electrical and/or electromagnetic energy of a particular waveform, intensity and/or time-course as needed to modulate the enteric, autonomic and/or somatic nervous systems to control colonic motility, fecal propulsion and defecation. In certain embodiments, the stimulator 40 is located external to the body while in other embodiments the stimulator 40 is located within a lumen of the body (e.g., rectum or colon), percutaneously implanted or fully implanted within the body.

The stimulation performed by the stimulator 40 is informed by the sensed physiological variates and the stimulation parameters of the stimulator are adjustable in real-time based on stimulus-elicited outcomes (e.g., sensor feedback) and/or user-entered dynamic parameters submitted through the programming device 60. Examples of dynamic parameters usable to control operation of the stimulator 40 can include, but are not limited to, stimulation frequency, stimulation amplitude, stimulation waveforms (e.g., pulse, sinusoid, burst, decaying, etc.). For example, a pulse train may ramp-up or ramp-down in frequency during a stimulation treatment. In certain embodiments, the stimulator 40 delivers stimulation through a single channel and a single pair of electrodes 50 while in other embodiments the stimulator delivers stimulation through a plurality of electrode pairs 50 wherein each of pair of electrodes 50 is independently through separate channels or, alternatively, commonly controlled through a common channel. In certain embodiments, the stimulator 40 simultaneously or sequentially generates different waveforms that can be correspondingly delivered through the same or different pairs of electrodes 50. For example, one waveform may serve to condition (facilitate, inhibit, or mask) nervous circuitry while other waveforms may modulate nervous or muscular tissue.

Stimulator output parameters can include, but are not limited to: (1) constant-current or constant-voltage outputs; (2) waveforms: sinusoidal, direct current (DC), impulses, pulses, multi-pulse trains or any combination thereof; (3) stimulation frequency: 0 Hz, or 1 Hz to 1000 Hz; (4) stimulation intensity: ≤200 mA or ≤200 V (peak-to-peak); (5) pulse width: 0.1 ms<10 ms; (g) train frequency: 0.5 to 20 trains per min.; and (6) train duration: 100≤2000 ms.

### E. Electrodes

One or more pairs of electrodes 50 are communicatively coupled with the stimulator 40 and are in contact with the body under treatment. The one or more pairs of electrodes operate to deliver stimulation to the enteric, autonomic, and/or somatic nervous system to control colonic motility, fecal propulsion and defecation.

The one or more pairs of electrodes 50 can be placed on the skin (transcutaneous electrodes), on the rectal or colonic wall (mucocutaneous electrodes) and/or be fully-implanted. The one or more pairs of electrodes 50 are typically fabricated from an electrically conductive metal or other electrically conductive material. Further, each of the electrodes 50 is embodied within a structure to assure reliable placement. In the instance of external placement, the electrodes 50 can be incorporated into a wearable garment or harness.

The one or more pairs of electrodes 50 deliver stimulation to the nerves and plexus within and surrounding the bowel and rectum including, but not limited to, afferent and efferent nerves of the somatic nervous system (e.g., pelvic and pudendal nerves, sensory afferents of the colon and rectum), autonomic nervous system (sympathetic and, sacral and vagal parasympathetic) and enteric nervous system (located within the colon wall). The one or more pairs of electrodes 50 can also deliver stimulation to cause direct contraction of the colonic wall musculature.

Electrodes 50 used for transcutaneous stimulation can be placed on the surface of the anterior abdomen proximate the hepatic and splenic flexures, and/or proximate the sigmoidal or rectosigmoid junctions, or at any position proximate the ascending, transverse or descending colon. Electrodes 50 that have been implanted can be placed proximate within the anterior abdomen proximate the nerves that innervate the hepatic and splenic flexures, the sigmoidal or rectosigmoid junctions, and/or the ascending, transverse or descending colon. Electrodes 50 that have been implanted can additionally or alternatively be placed on the posterior side of the body in the sacral foramen of S1, S2, S3, S4, and/or S5.

With regard to the enteric nervous system (ENS), the electrodes 50 can deliver transcutaneous or percutaneous stimulation to the myenteric and/or submucosal plexus. The myenteric plexus increases the tone of the gut and the velocity and intensity of contractions. The myenteric plexus is concerned with motility throughout the whole gut. Inhibition of the myenteric system helps to relax the sphincters—the muscular rings that control the flow of digested food or food waste. The submucosal plexus is involved in with local conditions and controls local secretion, absorption, and muscle movements. The mucosa and epithelial tissue associated with the submucosal plexus have sensory nerve endings that feed signals to both layers of the enteric plexus. These tissues also send information back to the sympathetic pre-vertabral ganglia, the spinal cord and the brain stem.

The one or more pairs of electrodes 50 can additionally or alternatively be used as a sensor 30 to detect physiological variates as described elsewhere herein.

### F. Programming Device

The programming device 60 is a device through which data is transmitted to and received from the controller 20 via wired or wireless communication. Example embodiments of the programming device 60 include a handheld device, a portable machine, a computer (e.g., smartphone, table, laptop, desktop, etc.), or any other device capable of providing the desired functionality described within the present disclosure by direct or remote interface to the controller 20.

In a direct interface configuration, operation of the programming device 60 can be directed by the controller 20 or, alternatively, the programming device 60 may employ its own controller or processor distinct from the controller 20. In an indirect interface configuration, the programming device 60 specifically includes its own controller or processor distinct from the controller 60. In certain embodiments, the programming device 60 includes a touch interface such as a touch screen, keyboard, stylus, one or more push buttons, and/or dials. In certain embodiments, the programming device 60 includes a visual display to display stimulation parameters and/or other data relative to the operation or desired operation of the system 10.

The programming device 60 functions to enable a user (e.g., patient or caregiver) to: (1) send commands to the controller 20; (2) adjust stimulation parameters which are transmitted to the controller 20 and stored in memory 23; (3) receive feedback from the sensors 30, stimulator 40 and/or electrodes 50 via the controller 20; (4) monitor usage of the system 10; and/or (5) receive/transmit data from the remote server 70.

### G. Data Analytics

In certain embodiments, one or more of the controller 20, the sensors 30, the stimulator 40, the electrodes 50 and the programming device 60 are in wired or wireless communication with a remote server 70 having memory enabling storage of current and historical operating data of one or more systems 10. The current and historical operating data can include, for example, a patient identifier, a system 10 identifier, stimulation parameters, and feedback from the sensors 30 and/or the electrodes 50 before and after stimulation. Additional data that can be associated with the operating data and used in analysis for determining appropriate treatment parameters include, but are not limited to, a patient's medical factors, medical history and types of spinal cord injuries or reasons for constipation.

The current and historical data can be analyzed for trends (e.g., stimulation parameters or other operating parameters such as the number, types, and/or placement of sensors and electrodes) that will help to inform a machine-learning algorithm that can be downloaded to the controller 20 for optimal stimulation and overall system 10 operation to obtain a bowel movement or another desired clinical outcome. For example, if someone has a cervical level spinal cord injury, they may need a different stimulation algorithm for success than someone that has a lumber level spinal cord injury. Other factors that cause constipation, such as slow transit of bowel, medication-induced constipation, opioid-constipation, may also have different stimulation parameters. The various types of constipation, including but not limited to slow transit, idiopathic, and opioid-induced constipation may all be addressed within the stored data and result in a plurality of algorithms each of which requires different types of stimulation.

In certain embodiments, the storage of current and historical data can be integrated with a user health and wellness app that also enables the user to enter self-report data such as dietary habits and receive recommendations for improving their condition. The health and wellness app is preferably integrated with a smart device that also enables the tracking of a patient's health outcomes, such as heart rate, sleep patterns, etc., which is data that can also be used to inform the machine-learning algorithm. The app, accessible via the smart device, can include a portal to allow for health coaching or connection with health care providers to interact through tele-medicine and/or access app technical support.

In certain embodiments, the server providing storage of current and historical system data can additionally provide a remote portal through which a patient's practitioner, provider, and/or a program coder can program the server and/or direct message a patient via text, audio and/or visual communication. This remote integration can help the patient to get the best outcome without direct programming of their personal system 10 or without an in-person physician office visit.

### H. Device Configurations

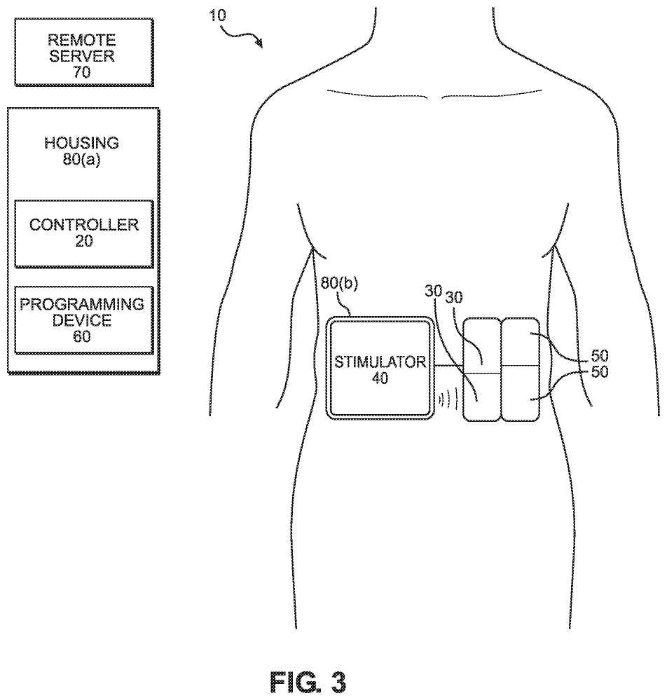

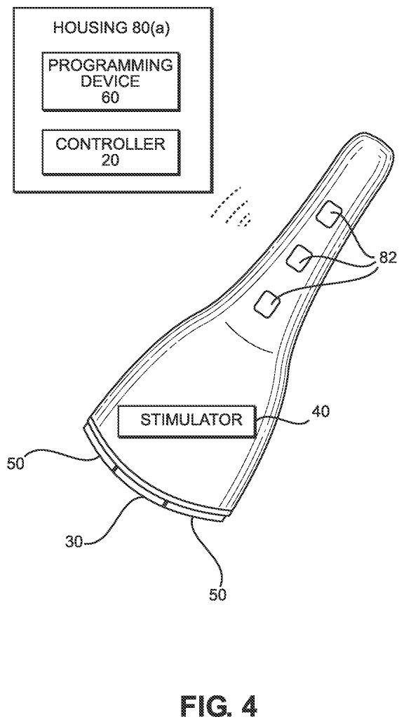

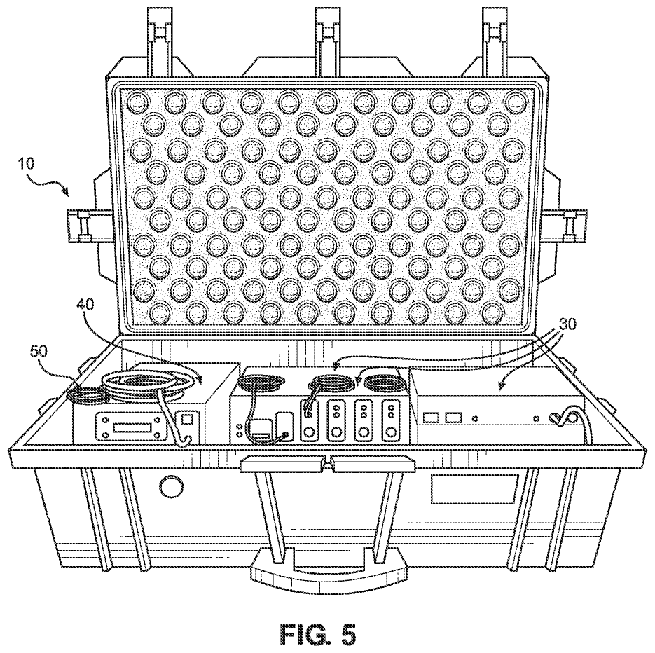

The components (e.g., controller 20, one or more sensors 30, stimulator 40, electrodes 50, and programming device 60) of the system 10 to acutely control colonic function, fecal propulsion, and defecation can be combined in various physical device configurations. For example, all or a portion of the components can be co-located within a singular device housing that may be handheld or otherwise portable. In another example, the programming device 60 is maintained in a device housing that is separate and distinct from another device housing in which the controller 20, one or more sensors 30, stimulator 40, and electrodes are colocated. In still another example, the controller 20 is provided with a housing that is separate and distinct from a programming device 60 housing and/or a stimulator housing. Other configurations are also possible. Notably, FIG. 3 illustrates a configuration where the programming device 60 and controller 20 are commonly housed in a housing 80(a) while the stimulator 40 is housed separately in a housing 80(b), which can be external to or implanted within the patient. FIG. 4 illustrates a configuration of a hand-holdable housing 80(c) containing the stimulator 40, one or more pairs of electrodes 50, and one or more sensors 30 with the programming device 60 and controller 20 are commonly housed in a housing 80(a). One or more push buttons 82 are provided on the hand-holdable housing 80(c). The push buttons 82 can be used to replicated functionality of the programming device 60 or be assigned alternative fuctionalities, e.g., turning on/off of the stimulator 40; other types of user interfaces may be used in place of the push buttons 82. FIG. 5 illustrates a prototype portable device configuration of the system 10 with one or more sensors 30, one or more pairs of electrodes 50, and the stimulator 40 identified; the controller 20 and programming device 60 are not present.

### I. Operation of Preferred Embodiment

In use, the system 10 to acutely control colonic function, fecal propulsion, and defecation is used for the treatment of acute or chronic constipation and/or neurogenic bowel dysfunction related to neurological injury or disorder such as spinal cord injury, multiple sclerosis, stroke, Parkinson's disease, spina bifida or amyotrophic lateral sclerosis, slow transit of the bowel, medication-induced constipation, opioid-induced constipation, post-surgical constipation, or aging.

General operation of the system 10 includes:

(a) Prior to defecation, positioning one more pairs of stimulation electrodes 50 and one or more sensors 30 proximate target nerves and structures; (electrodes may be external, partially implanted, or fully implanted)

(b) Delivering through electrodes 50 electrical or electromagnetic stimulation from the stimulator 40;

(c) Collecting sensor feedback (e.g., signals or measurements) and, optionally, user feedback, at the controller 20 to refine electrode placement and sensed response;

(d) Based on the sensor feedback:
(i) manually adjusting the placement of the one or more pairs of electrodes 50;
(ii) continuously adjusting in real-time the stimulation delivered through the electrodes 50 from the stimulator 40 based on the machine-learning algorithm for stimulation as determined from current and historical system data, the machine-learning algorithm being executed at a remote server in communication with the controller 20 or being executed at the controller 20;
(iii) adjusting the stimulation delivered through the electrodes 50 from the stimulator 40 responsive to feedback from the one or more sensors 30 (e.g., real-time closed loop control); and/or
(iv) adjusting the stimulation delivered through the electrodes 50 from the stimulator 40 by adjusting system operating parameters (e.g., stimulation parameters) via user-entry of stimulation parameter settings at the programming device 60;

(e) Repeat steps (c)-(d) until productive defecation is achieved or a desired outcome is produced.

(f) Reuse the system 10 as needed to produce defecation or the desired outcome utilizing one or more of the steps described above.

In a preferred embodiment, the system 10 is external to the body, battery-operated, rechargeable and provides wireless communication between the programming device 60 and the controller 20 to facilitate fecal propulsion, colonic function, and defecation. The stimulator 40 is activated to modulate parasympathetic and enteric activity, and colonic musculature by delivering transcutaneous stimulation via electrodes 50 proximate the hepatic and/or splenic flexures and/or via electrodes 50 proximate the sigmoidal or rectosigmoid junctions. The stimulation parameters of the stimulator 40 responsively adjust to sensed respiration and sensed electrical and/or acoustic signals that describe colonic contraction and cause fecal propulsion. A single stimulation channel of the stimulator 40 for a single pair of electrodes 50 is used and delivers a composite stimulation waveform that is designed to condition atrophied enteric nervous and muscular tissue and to modulate targeted tissues to elicit colonic contraction.

The stimulation waveform used for modulation includes bursts of electrical pulses (e.g., constant-voltage square-waves, 100 μs pulse width, amplitudes <100 V, 100 Hz stimulation frequency, 100 pulses per burst) occurring 2.5 times per minute and a conditioning stimulus including high-amplitude (<100 V) impulses occurring irregularly between 0.5 and 20 times per minute. The stimulation amplitude, frequency, pulse-width, and burst-rate would increase and decrease depending on user feedback, respiratory patterns, and recorded markers of colonic activity. Electrocardiogram signals are used to recognize signs of autonomic dysreflexia (AD) and stimulation is terminated if the signs of AD are met.

Treatment of persons having reflexic bowel with the system 10 includes pacing the disorganized, arhythmical sacral parasympathetic activity that is responsible for colonic propulsion. The one or more sensors 30 record colonic electromyogram signals and the controller 20 uses them as feedback to inform stimulation parameters. The stimulator 40 drives reflexive, coordinated parasympathetic activity along the entire length of the bowel by activating the low-threshold sensory afferents that innervate the rectum and colon. The activation of sensory afferents in the rectum and bowel reflexively activates sacral parasympathetic outflow and colonic contraction. Other sensors 30 (e.g., goniometer, strain gauges, accelerometers, gyroscopes) of the system 10 are used to mark bowel activity and their feedback is utilized by the controller 20, via the stimulator 40, to drive sacral parasympathetic activity; the other sensors 30 include or more of those that measure intestinal movements or sounds caused by the movement, electrocardiogram signals, respiratory patterns, impedance and galvanic skin response.

In treatment of patients with neurogenic bowel with unreliable spinal reflexes, the system 10 operates by directly stimulating, via stimulation delivered through the electrodes 50 from the stimulator 40, the sacral parasympathetic efferent fibers, enteric fibers and intestinal wall musculature, without relying on spinal reflexes. The system 10 delivers a stimulation paradigm that includes multi-pulse trains of electrical stimulation, delivered in a burst waveform, to the hepatic or splenic fixtures and/or to the sigmoidal and rectosigmoid junctions. The timing of the bursts and the parameters (e.g., amplitude, frequency, duration, etc.) of the multi-pulse stimuli within each burst are informed by sensed activity (sensed through one or more sensors 30) such as colonic electromyography, acoustic signs of mechanical contractions, electrocardiogram signals, impedance, galvanic skin response and/or respiration.

In treatment of patients with neurogenic bowel having unreliable spinal reflexes, an alternative stimulation approach can also be used by the system 10. The alternative stimulation approach includes overlaying the burst stimulation waveform, which is delivered via the electrodes 50 from the stimulator 40, used to modulate nervous and muscular tissue with a second stimulation waveform that conditions the parasympathetic and enteric effector fibers and colonic musculature. The second conditioning stimulation waveform helps to control the atrophied (reduced activation threshold) or super-sensitive (increase activation threshold) excitable tissues during modulation. The second conditioning stimulation waveform is delivered through a same electrode pair as delivers the first burst stimulation waveform or through an independently controlled second electrode pair.

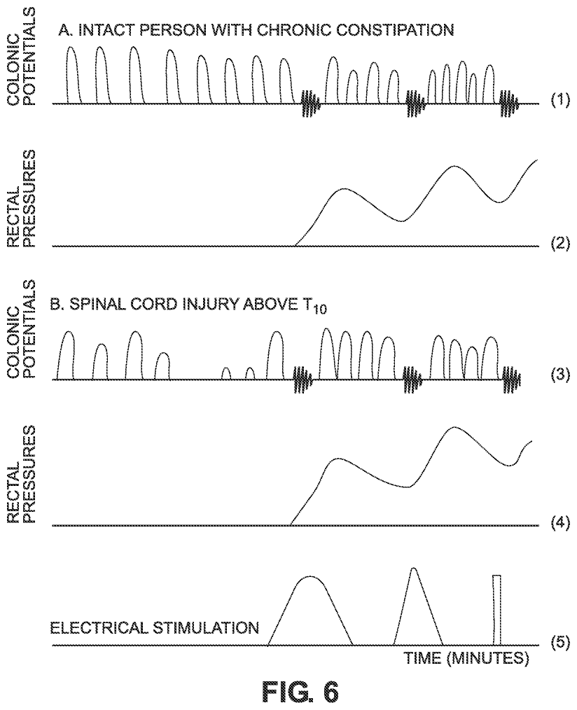

Referring to FIG. 6, lines 1 and 2 illustrate colonic potential and rectal pressure data, respectively, of a person with chronic constipation and with an intact nervous system while line 5 illustrates an example stimulation paradigm of the system 10 for treating the chronic constipation. Lines 3 and 4 of FIG. 6 illustrate colonic potential and rectal pressure, respectively, of a person with a spinal cord injury above T10 who is suffering chronic constipation due to neurogenic bowel dysfunction due to the spinal cord injury; the example stimulation paradigm of line 5 can also be used to treat the neurogenic bowel dysfunction.

In view of the above a system, device and method to sense physiological variates (nervous, mechanical or acoustic) and to deliver adaptable electrical stimulation to acutely control colonic motility, fecal propulsion and defecation in persons with constipation responsive to sensed markers of colonic activity from the enteric, autonomic and/or somatic nervous systems is described.

The system, device and method provide for the generation of energy of a particular waveform, intensity and/or time-course to modulate the targeted nervous system.

The system, device and method provide for stimulation parameters that accommodate the various types of tissues that are modulated (e.g., sensory afferents, autonomic efferents, colonic musculature), and are adaptable to change the stimulation parameters to acutely drive the circuitry from rest to productive defecation.

The system, device and method provide for the delivery of one or more stimulation waveforms that can be simultaneously and/or sequentially delivered to a patient to condition (e.g., facilitate, inhibit, mask, block) some tissues while modulating others.

The system, device and method provide for adaptive stimulation therapy that can be driven through a data analytic repository and machine learning algorithm.

Unless otherwise defined, all technical and scientific terms used herein have the same meaning as commonly understood by one of ordinary skill in the art to which this invention belongs. Although methods and materials similar to, or equivalent, to those described herein can be used in the practice or testing of the various embodiments of the present disclosure, suitable methods and materials are described above. All patent applications, patents, and printed publications cited herein are incorporated herein by reference in their entireties, except for any definitions, subject matter disclaimers or disavowals, and except to the extent that the incorporated material is inconsistent with the express disclosure herein, in which case the language in this disclosure controls. The various embodiments of the present disclosure may be embodied in other specific forms without departing from the spirit or essential attributes thereof, and it is therefore desired that the various embodiments in the present disclosure be considered in all respects as illustrative and not restrictive. Any headings utilized within the description are for convenience only and have no legal or limiting effect.

What is claimed is:

1. A system for fecal propulsion comprising:
a controller executing instructions stored in a memory;
one or more sensors communicatively coupled to the controller, wherein the one or more sensors sense at least one marker of colonic activity from one or more of: the enteric nervous system, the autonomic nervous system, and the somatic nervous system of a patient;
a stimulator communicatively coupled to the controller; and
one or more pairs of electrodes operatively coupled to the stimulator,
wherein the controller directs the stimulator to deliver stimulation, via the one or more pairs of electrodes to one or more of: the enteric nervous system, the autonomic nervous system, and the somatic nervous system, responsive to the at least one marker of colonic activity to produce fecal propulsion within the patient.

2. The system of claim 1, wherein the one or more sensors additionally sense a physiological variate different from the at least one marker of colonic activity and wherein the controller directs the stimulator to deliver stimulation responsive to both the physiological variate and the at least one marker of colonic activity.

3. The system of claim 2, wherein the physiological variate includes one or more of: intestinal movements, sounds caused by intestinal movements, electrocardiogram signals, respiratory patterns, muscle tone, autonomic tone, pressor movements, relaxation movements, gross movements, impedance and galvanic skin response.

4. The system of claim 1, wherein the stimulation comprises electric energy.

5. The system of claim 1, wherein the stimulation comprises electromagnetic energy.

6. The system of claim 1, wherein the stimulation comprises a stimulation waveform.

7. The system of claim 1, wherein the stimulation waveform is determined by the controller based on a predetermined algorithm that is responsive to the at least one marker of colonic activity.

8. The system of claim 7, wherein the predetermined algorithm comprises a machine-learning algorithm based on historical operating data of the system.

9. The system of claim 1, wherein the stimulation comprises at least two different waveforms delivered by the one or more pairs of electrodes.

10. The system of claim 9, wherein the at least two different waveforms are delivered simultaneously.

11. The system of claim 10, wherein a first of the at least two different waveforms conditions one or more of: the enteric nervous system, the autonomic nervous system, and the somatic nervous system, and wherein a second of the at least two different waveforms modulates nervous and/or muscular tissue.

12. The system of claim 9, wherein the at least two different waveforms are delivered sequentially.

13. The system of claim 1, wherein at least one pair of the one or more pairs of electrodes comprise external electrodes placed on the skin of the patient.

14. The system of claim 13, wherein the external electrodes placed on the skin of the patient are maintained in position with a wearable garment.

15. The system of claim 1, wherein at least one pair of the one or more pairs of electrodes is implanted within the patient.

16. A system for fecal propulsion comprising:
a programming device;
a controller in communication with the programming device, the controller executing instructions stored in a memory;
one or more sensors communicatively coupled to the controller, wherein the one or more sensors sense at least one marker of colonic activity from one or more of: the enteric nervous system, the autonomic nervous system, and the somatic nervous system of a patient;
a stimulator communicatively coupled to the controller; and
one or more pairs of electrodes operatively coupled to the stimulator,
wherein the controller directs the stimulator to deliver stimulation, via the one or more pairs of electrodes to one or more of: the enteric nervous system, the autonomic nervous system, and the somatic nervous system, responsive to the at least one marker of colonic activity to produce fecal propulsion within the patient.

17. The system of claim 16, wherein the programming device is in direct communication with the controller.

18. The system of claim 16, wherein the programming device is in remote communication with the controller.

19. The system of claim 16, wherein the programming device enables adjustment of one or more stimulation parameters that define the stimulation.

20. The system of claim 16, wherein the programming device receives feedback from one or more of: the one or more sensors, the stimulator and the one or more pairs of electrodes.
